(19) United States
(12) Patent Application Publication
(10) Pub. No.: US 2021/0361432 A1
(43) Pub. Date: Nov. 25, 2021

(54) Penile Implant Device and Method

(71) Applicant: Menova International, Inc., Beverly Hills, CA (US)

(72) Inventor: James J. Elist, Beverly Hills, CA (US)

(73) Assignee: Menova International, Inc., Beverly Hills, CA (US)

(21) Appl. No.: 16/882,167

(22) Filed: May 22, 2020

(51) Int. Cl. A61F 2/26; A61B 18/12

(52) U.S. Cl. CPC A61F 2/26; A61B 18/12; A61F 2002/0081; A61F 2220/0091; A61F 2230/0069

(57) ABSTRACT

A penile implant enhancement device and method is provided. The penile implant device comprises an elongated cylindrical sleeve that is configured to encircle a shaft of a penis and an external layer. The elongated cylindrical sleeve is defined by one or more edges, surfaces, or layers, including a proximal edge, a distal edge, a first side, a second side, a top surface, and a bottom surface, and the external layer is configured to cover the one or more edges, surfaces, or layers of the sleeve to form one or more covered edges, surfaces, or layers. The covered edges, surfaces, or layers are configured to align with the penis. The method comprises the steps of administering one or more anesthetic agents; cutting a transverse incision above the pubic symphysis; clamping a lower edge of the transverse incision; dissection through the one or more layers of subcutaneous tissue to expose tunica albuginea; everting the penis; creating a pocket between the tunica albuginea and the skin; providing the aforementioned penile implant device; suturing the covered edges, surfaces, or layers to the penis; trimming the external layer, reverting the penis; and closing the transverse incision.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to surgically implanted devices and methods and, more particularly, to surgically implanted prostheses for the enhancement of appearance and operation of the penis while avoiding complications commonly associated with other prostheses and procedures.

### BACKGROUND

Penile anatomy generally comprises a shaft portion and a head portion, otherwise known as the glans penis. The glans penis protrudes outward over the shaft portion, thereunder forming the coronal sulcus. Along the length of the shaft, two columns of tissue form the corpus cavernosum and one forms the corpus spongiosum, which are all enveloped by tunica albuginea. Beneath the skin, generally two layers of fascia, Buck's fascia and Dartos fascia, surrounds the tunica albuginea. Moreover, other subcutaneous tissue, including subcutaneous areolar tissue, is situated between the skin and the tunica albuginea. At the proximal end, the penis is supported by the suspensory ligament, which attaches to the pubic symphysis.

While a large sect of men may desire penile elongation or enhancement, surgical penile cosmetic correction has generally been reserved for men who had a penis that was buried in the suprapubic pannus or a micropenis (an adult penis that is considered abnormally small). Methods for these patients have focused on extending the length of the penis outside the body plane, such as by cutting the suspensory ligament and implanting autologous fat or artificial grafts. However, these procedures can cause undesired outcomes and complications.

For instance, because many penile elongation operations involve cutting of the suspensory and/or fundiform ligaments of the penis, patients may experience penile shortening resulting from the freely hanging penis reattaching to the pubic bone higher, loss of sensation, angling of the penis downward due to lack of support, and hypertropic scarring of wounds. By way of further example, penile deformity, paradoxical penile shortening, scarring, granuloma formation, migration of injected material, and sexual dysfunction are frequently reported after alternate procedures. Further, low levels of short- and long-term patient satisfaction have also been commonly experienced. Thus, there exists a need for surgical penile cosmetic correction that avoids the aforementioned, and other, negative side effects while still maintaining a desirable aesthetic outcome.

### SUMMARY

The present disclosure is directed to penile implant enhancement devices and methods that improve penile appearance by expanding girth and/or length of the penis outside the body. More particularly, the penile implant device may cosmetically correct the penises of patients who present with small penis perception, buried penis, micropenis, and other related diagnoses, while avoiding adverse changes in penile function. Further, the penile implant device and method may improve a patient's self-confidence and self-esteem.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, the penile implant device may comprise an elongated cylindrical sleeve and an external layer. The elongated cylindrical sleeve may be configured to encircle a shaft of a penis, either partially or wholly. The external layer may provide rigidity and may be configured to cover one or more edges, surfaces, or layers of the sleeve and form one or more covered edges, surfaces, or layers to align with and secure to the penis. More specifically, in certain exemplary embodiments, a midline of the external layer may join to and cover a distal edge of the sleeve so as to form a double layer. Moreover, the elongated cylindrical sleeve may support the penis shaft and permit enlargement of the same during erection, while the external layer may ensure security of the device to the penis.

According to certain embodiments, the elongated cylindrical sleeve may further comprise a hinge disposed along a longitudinal midline of the sleeve and a first side and a second side, which may abut the hinge. In other embodiments, the elongated cylindrical sleeve may comprise at least one hinge and a plurality of hingedly joined sides, each of the hingedly joined sides abutting along one of the at least one hinge such that the plurality of hingedly joined sides may align sequentially. In alternate embodiments, the elongated cylindrical sleeve may comprise a unitary body, that is, a single piece. For purposes of brevity, the remainder of this disclosure will describe in more detail embodiments wherein the elongated cylindrical sleeve may comprise exactly two sides, that is, the first side and the second side, as shown in the Figures.

As mentioned above, the elongated cylindrical sleeve may be peripherally defined by the one or more edges, surfaces, or layers, such as a proximal edge, a distal edge, a first lateral edge, and a second lateral edge. Moreover, the sleeve may be further defined by one or more surfaces, such as a top surface and a bottom surface. In some embodiments, the elongated cylindrical sleeve, and therefore the device, may be in an open state such that the first side and the second side have rotated in an open direction about the hinge. In such embodiments, the first lateral edge and the second lateral edge may rotate apart from one another. For example, the elongated cylindrical sleeve may be in the open state when the penis is erect. In alternate embodiments, the sleeve may be in a closed state such that the first side and the second side have rotated in a closed direction about the hinge. In such embodiments, the first lateral edge of the first side may physically contact the second lateral edge of the second side but may not secure to the second lateral edge. For instance, the sleeve may be in the closed stated when the penis is flaccid.

In certain embodiments, the elongated cylindrical sleeve may be formed out of medical-grade silicone and/or medical-grade polyester. Alternatively, the elongated cylindrical sleeve may be formed out of other medical-grade materials as well. According to some embodiments, the elongated cylindrical sleeve may be formed out of a gel-like material or may comprise saline, marbles, or other materials. In some embodiments, the elongated cylindrical sleeve may be smooth along its length. In other embodiments, the elongated cylindrical sleeve may comprise one or more grooves, lines, shapes, patterns, or symbols.

Moreover, the elongated cylindrical sleeve may be formed out of one or more internal layers, which may be formed out of mesh or other materials that may provide rigidity. In other embodiments, the one or more internal layers may be formed of a stretchable material. For instance, the stretchable material may comprise an accordion stretch material. In some embodiments, the one or more internal layers may be formed of one or more air pockets. Further, the elongated cylindrical sleeve may comprise one or more anchor points for sutures so as to secure the device to the penis. In further embodiments, the elongated cylindrical sleeve may be formed of one or more of an antimicrobial material and an antibacterial material.

In some embodiments, the elongated cylindrical sleeve may be unfixed from the penile shaft and therefore, may remain free to expand and contract during erection, intercourse, and flaccidity. Additionally, the size, including length and width, of the elongated cylindrical sleeve may vary. Moreover, the size of the elongated cylindrical sleeve may vary depending on the natural size and shape of the patient's penis. In other embodiments, the size of the elongated cylindrical sleeve may vary depending on the desires of the patient. In this way, the elongated cylindrical sleeve may allow for customization based on patient needs or desires. The elongated cylindrical sleeve may also be formed out of a plurality of segments along its length, that is, from the distal edge to the proximal edge. In such embodiments, additional flexibility may be provided. In even further embodiments, the elongated cylindrical sleeve may be formed of a stretchable material, such as an accordion stretch material.

According to some embodiments, the elongated cylindrical sleeve may also comprise a press-rib disposed along the longitudinal midline of the sleeve. In certain embodiments, the press-rib may extend only partially the length of the longitudinal midline while in others, the press-rib may extend the full length of the longitudinal midline. When the first side and the second side rotate outwardly, such as during an erection, the press-rib may be forced to apply pressure to a deep dorsal vein of the penis, thereby preventing reverse blood flow and maintaining the erection. The press-rib may be shaped as a teardrop, triangle, cylinder, or other convenient and desirable shape. The press-rib may also be formed as a singular piece or may be divided into multiple pieces for increased flexibility.

The external layer may cover the one or more edges, surfaces, or layers of the elongated cylindrical sleeve. Once the external layer covers the one or more edges, surfaces, or layers, the one or more covered edges, surfaces, or layers may anchor the device to the penis along the tunica albuginea. In certain embodiments, such as that shown in the Figures, the external layer may be configured to cover the distal edge of the elongated cylindrical sleeve to form a covered distal edge. The covered distal edge may be configured to align with a coronal sulcus of the penis. In certain exemplary embodiments, the covered distal edge may be formed to secure to the coronal sulcus, such as by suturing. In some embodiments, the covered distal edge may secure to the coronal sulcus, and more particularly, the tunica albuginea, using one or more nonabsorbable surgical sutures. For example, polyester sutures may be used. In other embodiments, one or more absorbable surgical sutures or a combination of nonabsorbable and absorbable surgical sutures may be used to secure the covered distal edge to the coronal sulcus.

In addition, the external layer may be formed as a sheet. While the external layer may be shown and described throughout this disclosure as a mesh layer, a person of ordinary skill in the art will recognize that any material which provides rigidity may be used as the external layer. In some embodiments, the external layer be formed of a soft mesh material, such as polypropylene. In alternative embodiments, the external layer may be formed out of other biocompatible materials, such as bovine tissue, silicone, marbles, or strings. However, one of ordinary skill in the art will recognize other biocompatible materials or even non-biocompatible materials. In some embodiments, the external layer may be imbedded within the elongated cylindrical sleeve. The external layer may enable tissue ingrowth and therefore, may better allow the patient to acclimate to the device. The external layer may also diminish the possibility of perforation or erosion of the glans penis. Moreover, in some embodiments, the external layer may be formed of one or more of an antimicrobial material and an antibacterial material.

In some embodiments, the elongated cylindrical sleeve may be contiguously joined with the external layer. In such embodiments, the sleeve and the external layer may comprise a single-bodied penile implant device. In other embodiments, the elongated cylindrical sleeve and the external layer may be separate. In these embodiments, the sleeve and the external layer may be joinable along the one or more edges, surfaces, or layers of the sleeve. In some embodiments, the external layer may be joinable along the distal edge of the sleeve. More specifically, the external layer may cover the sleeve at a midline of the external layer and may be secured to the sleeve and the penis using sutures or an equivalent material.

In some embodiments, the penile implant device may further comprise one or more antimicrobial layers and/or one or more antibiotic layers. In certain embodiments, the one or more antimicrobial layers and/or one or more antibiotic layers may be secured to the elongated cylindrical sleeve. In alternate embodiments, the one or more antimicrobial layers and/or one or more antibiotic layers may be secured to the external layer. In still other embodiments, the one or more antimicrobial layers and/or one or more antibiotic layers may be secured to the external layer or the elongated cylindrical sleeve while the penile implant device is being implanted within the patient's penis.

In one embodiment of the present invention, a method involving the aforementioned penile implant device may be used to enhance the cosmetic appearance of the penis with minimal risk for post-operative complications. The method may comprise the steps of: administering one or more anesthetic agents; cutting an at least ¼-inch transverse incision above the pubic symphysis; clamping a lower edge of the transverse incision to expose one or more layers of subcutaneous tissue; dissecting through the one or more layers of subcutaneous tissue to expose tunica albuginea; everting the penis; creating a pocket between the tunica albuginea and the skin; providing a penile implant device defined by an elongated cylindrical sleeve and an external layer covering one or more edges, surfaces, or layers of the sleeve to form one or more covered edges, surfaces, or layers; suturing the one or more covered edges, surfaces, or layers to the penis; trimming the external layer; reverting the penis; and closing the transverse incision.

The one or more anesthetic agents may comprise general anesthesia, so as to render the patient unconscious during the procedure, a local anesthetic agent, and/or spinal anesthesia. Additionally, in some embodiments, the one or more anesthetic agents may comprise both general anesthesia and the anesthetic agent. For instance, the local anesthetic agent may comprise lidocaine, bupivacaine, a mixture of lidocaine and bupivacaine, or any other long-acting local anesthetic agent or combination thereof. Moreover, in some embodiments, the local anesthetic agent may be injected around the base of the penis. In further embodiments, the one or more anesthetic agents may comprise general twilight anesthesia, that is, general anesthesia that is administered to the patient in a mild dose so as to induce anxiolysis. Spinal anesthesia may provide a sensory blockade during the procedure.

To begin the operation, cutting the at least ¼-inch transverse incision may comprise cutting the incision 2-3 centimeters above the pubic symphysis, that is, approximately 1 inch above the suprapubic bone. Once cut, the transverse incision may be defined by an upper edge and a lower edge. Cutting the transverse incision may expose the first layer beneath the skin, Dartos fascia. In addition, cutting the transverse incision above the pubic symphysis may open a suprapubic space at the base of the penis, allowing access to the penis.

One or more Allis clamps may be used to clamp the lower edge of the transverse incision to expose Dartos fascia. In alternate embodiments, other surgical instruments may be used to hold or grasp the lower edge of the transverse incision, such as another surgical clamp, tenaculum, or Cushing forceps. In certain embodiments, two Allis clamps may be used to clamp the lower edge of the incision. Importantly, any surgical instrument used to clamp the lower edge of the incision may be operative to withstand the weight of heavy tissue, such as that found in the suprapubic region.

Scissors, an electrocautery, or other surgical instruments may be used for dissecting through the one or more layers of subcutaneous tissue. The one or more layers of subcutaneous tissue may comprise Buck's fascia, Dartos fascia, and areolar tissue. Any and all points of bleeding may be electrocauterized using the electrocautery. In certain exemplary embodiments, a surgical instrument operative for sharp dissection may be used to dissection through the subcutaneous tissue. Further, dissecting through the one or more layers of subcutaneous tissue may also comprise releasing tissue from the suspensory ligament of the penis, while preserving attachment of the suspensory ligament. In such embodiments, preserving attachment of the suspensory ligament may prevent the device from sliding back beneath the pubic symphysis.

In certain exemplary embodiments, creating a pocket between the tunica albuginea and the skin may comprise creating a pocket between Buck's fascia and Dartos fascia. In some embodiments, the pocket between Buck's fascia and Dartos fascia may be created by dissecting through the areolar tissue between Buck's fascia and Dartos fascia. The pocket between Buck's fascia and Dartos fascia may be extended the entire length of the penile shaft, that is, from a proximal edge of the shaft to the glans penis. According to some embodiments, the pocket may not comprise the ventral urethral area of the shaft and instead, may only comprise a ¾ circumferential dissection around the shaft.

In some embodiments, after the pocket is created between the tunica albuginea and the skin, the coronal sulcus of the penis may be defined. In particular, the tissue around the coronal sulcus of the penis may be sharply dissected. The coronal sulcus may be accessed as a result of previously everting the penis. Defining the coronal sulcus may further comprise releasing areolar tissue therefrom. In some embodiments, defining the coronal sulcus may allow for subsequent suturing of the device to the coronal sulcus. Indeed, defining the coronal sulcus may allow a distal edge of the device to be nestled adjacent to the junction of the shaft and the glans penis.

Providing the penile implant device, which may be defined by the elongated cylindrical sleeve and the external layer, may further comprise providing the elongated cylindrical sleeve and the external layer separately and then, covering the external layer over one or more edges, surfaces, or layers of the sleeve to from the one or more covered edges, surfaces, or layers. In other embodiments, providing the penile implant device may further comprise providing the sleeve and the external layer as a single-bodied device. In such embodiments, the sleeve and the external layer may be sutured or otherwise secured to one another prior to surgery. Alternatively, the external layer may cover the proximal edge, the first lateral edge, the second lateral edge, the top surface, the bottom surface, or a combination of the foregoing edges, surfaces, or layers.

Nonabsorbable surgical sutures may be used for suturing the covered edges, surfaces, or layers to the tunica albuginea. According to certain exemplary embodiments, the nonabsorbable surgical sutures may comprise polyester sutures. In other embodiments, the sutures may comprise silk, nylon, or stainless-steel sutures. In alternate embodiments, one or more absorbable sutures or a combination of absorbable and nonabsorbable sutures may be used. Moreover, as previously discussed, in some embodiments, the external layer may cover the one or more edges, surfaces, or layers of the elongated cylindrical sleeve to form one or more covered edges, surfaces, or layers. In such embodiments, each of the one or more covered edges, surfaces, or layers may secure to the tunica albuginea.

After suturing the one or more covered edges, surfaces, or layers to the tunica albuginea, the external layer may be trimmed. In some embodiments, scissors or other cutting instruments may be used to trim the external layer. Further, in certain embodiments, the external layer may be trimmed so as to leave 1 centimeter of the external layer around each of the sutures.

Finally, to complete the surgery, the penis may be reverted and the transverse incision may be closed. Closing the transverse incision may further comprise suturing the transverse incision. Alternatively, closing the transverse incision may comprise gluing the transverse incision using medical-grade skin glue. A person of ordinary skill in the art will recognize other acceptable means of closing the transverse incision.

In further embodiments, prior to the aforementioned steps, the method may further comprise preparing a surgical site. The surgical site may comprise the penis and the surrounding area which may be affected or touched during surgery. Preparation of the surgical site may further comprise draping the surgical site with an antimicrobial drape and measuring preoperative penile circumference and length. The antimicrobial drape may reduce the risk of infection of the surgical site by immobilizing bacteria and providing continuous antimicrobial activity, thereby disallowing bacteria on the skin to infect the surgical site.

In even further embodiments, throughout the method, the method may further comprise applying one or more antibiotics. According to certain embodiments, the one or more antibiotics may comprise a triple antibiotic solution mixed with rifampicin. In alternate embodiments, the one or more antibiotics may comprise a triple antibiotic solution mixed with minocycline or a combination of rifampicin and minocycline. However, other antibiotics or antibiotic solutions may be applied in accordance with the method. Indeed, a person of ordinary skill in the art will recognize other such appropriate antibiotics. The one or more antibiotics may inhibit or slow the growth of bacteria in the surgical site. Moreover, applying the one or more antibiotics may further comprise irrigating or lavaging the surgical site with the one or more antibiotics.

In still further embodiments, after the aforementioned steps, the method may further comprise installing a surgical drain. The surgical drain may collect fluid that builds up inside the body in a surgical or traumatized area. The surgical drain may render less likely infection and other complications arising from this fluid buildup. In some embodiments, the surgical drain may comprise a Jackson-Pratt drain. In alternate embodiments, the surgical drain may comprise a Penrose drain, a Redivac drain, negative pressure wound therapy, a Davol, a pigtail drain, or any other type of drain or instrument for removing collected fluid from the surgical site.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

FIG. 1 shows a perspective view of an embodiment of the penile implant device.

FIG. 2 shows a cross-sectional view of an embodiment of the penile implant device.

FIG. 3 shows a cross-sectional view of an embodiment of the penile implant device.

FIG. 4 shows a perspective view of an embodiment of the penile implant device.

FIG. 5 shows an exploded perspective view of an embodiment of the penile implant device.

FIG. 6 shows an embodiment of the penile implant method.

The disclosed embodiments may be better understood by referring to the figures in the attached drawings, as provided below. The attached figures are provided as non-limiting examples for providing an enabling description of the method and system claimed. Attention is called to the fact, however, that the appended drawings illustrate only typical embodiments of this invention and are therefore not to be considered as limiting of its scope. One skilled in the art will understand that the invention may be practiced without some of the details included in order to provide a thorough enabling description of such embodiments. Well-known structures and functions have not been shown or described in detail to avoid unnecessarily obscuring the description of the embodiments.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

A penile implant device and method is provided that, in some embodiments, may cause enhanced penile appearance while avoiding adverse changes in sexual and other functions. In addition to cosmetically correcting the penis, the penile implant device may improve a patient's self-confidence, self-esteem, and overall self-satisfaction. The penile implant device may comprise an elongated cylindrical sleeve configured to encircle a shaft of a penis and an external layer configured to align with and secure to the tunica albuginea of the penis.

While the device and method is described herein as comprising only the elongated cylindrical sleeve and the external layer, either together or as separate components, a person of ordinary skill in the art will recognize that the invention discussed herein should not be limited to the specific exemplary embodiments provided. Indeed, the elongated cylindrical sleeve may comprise a plurality of hingedly joined sides 116a, 116b. In other embodiments (not shown), the elongated cylindrical sleeve may comprise a unitary body. However, in embodiments illustrated in the FIGS. herein, the elongated cylindrical sleeve 110 may comprise a first side 116a and a second side 116b disposed along one hinge 118. Moreover, the external layer 120 may comprise one or more of external layers that may be configured to align with one or more edges, surfaces, or layers of the sleeve. As shown herein, the external layer 120 may be configured to align with a distal edge 114 of the sleeve 110.

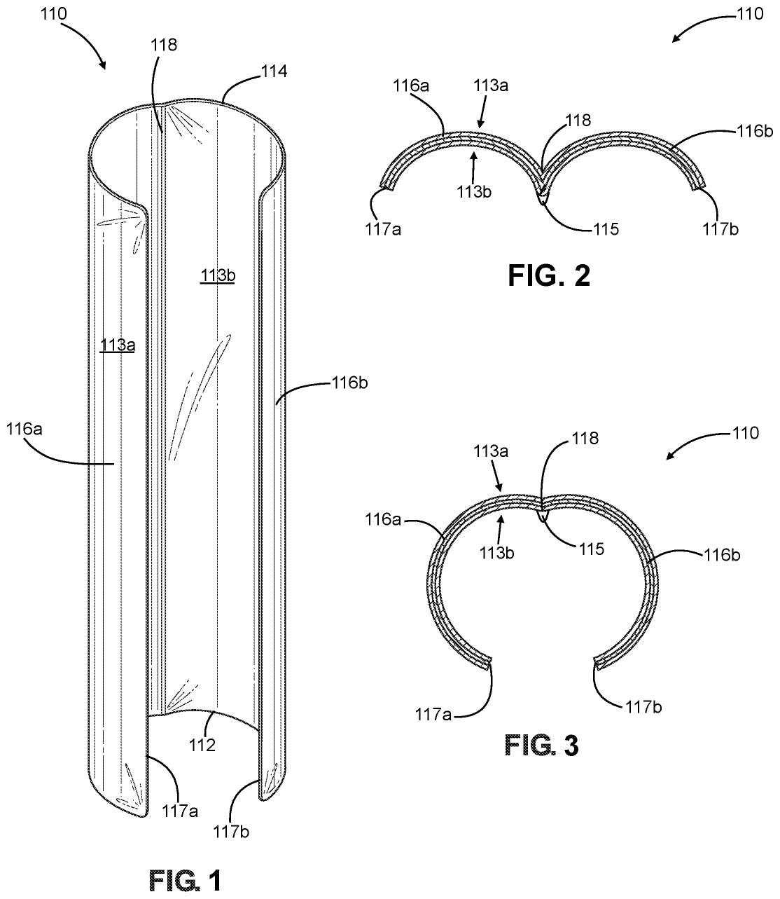

More particularly, according to one embodiment shown FIG. 1, the elongated cylindrical sleeve 110 may be defined by a proximal edge 112, the distal edge 114, a top surface 113a, a bottom surface 113b, the first side 116a and the second side 116b. In certain embodiments, the proximal edge 112 of the sleeve 110 may correspond to a proximal edge of the penis, that is, the edge closest to a base of the penis and the patient's torso. Similarly, the distal edge 114 of the sleeve 110 may correspond to a distal edge of the penis, that is, the edge closest to the glans penis and furthest from the patient's torso. In other words, the proximal edge 112 of the sleeve 110 may align with a base of the penis while the distal edge 114 of the sleeve 110 may align with the coronal sulcus along the glans penis. Moreover, the top surface 113a may correspond to a dorsal side of the penis, while the bottom surface 113b may correspond to the ventral side of the penis. Thus, the bottom surface 113b may contact and align with the dorsal side of the penis while the top surface 113a may remain exposed. In some embodiments, the proximal edge 112 may remain unattached to the penis and therefore, may freely expand and contract during erection, intercourse, and flaccidity. In alternate embodiments, the proximal edge 112 may secure to the penile base.

Referring now to FIGS. 2-3, the first side 116a and the second side 116b of the elongated cylindrical sleeve 110 may be joined together along the hinge 118. The hinge 118 may be disposed along a longitudinal midline of the sleeve 110 such that the first side 116a may be positioned along one half of the midline while the second side 116b may be positioned complimentarily along the other half of the midline. The first side 116a and the second side 116b may be further defined by a first lateral edge 117a and a second lateral edge 117b, respectively. The first and second lateral edges 117a, 117b may be positioned opposite the longitudinal midline of the sleeve 110 and therefore, the hinge 118. Indeed, in some embodiments, the hinge 118 may be equidistant between the first lateral edge 117a and the second lateral edge 117b. Moreover, the first lateral edge 117a may run parallel to the second lateral edge 117b. In certain embodiments, the first lateral edge 117a may contact, but not secure to, the second lateral edge 117b.

According to certain embodiments, such as that shown in FIG. 2, when the penis is erect, the first side and the second side 116a, 116b may rotate outwardly, that is, in an open direction, around the hinge 118. Indeed, in such embodiments, the penis may require greater space to expand while engorging with blood. In these embodiments, the first lateral edge 117a may move further apart from the second lateral edge 117b. On the other hand, as shown in FIG. 3, when the penis is flaccid, the first side and the second side 116a, 116b may rotate inwardly, that is, in a closed direction, around the hinge 118. In such embodiments, the first lateral edge 117a may move closer toward the second lateral edge 117b. In further flaccid embodiments, the first lateral edge 117a may contact the second lateral edge 117b.

In some embodiments, such as that shown in FIGS. 2-3, the elongated cylindrical sleeve 110 may also comprise a press-rib 115 disposed along a bottom surface of the sleeve 110 along the longitudinal midline. Indeed, the press-rib 115 may be disposed opposite the hinge 118 along the longitudinal midline of the sleeve 110. While the press-rib 115 is shown in FIGS. 2-3 as extending the entire length of the longitudinal midline, in other embodiments, the press-rib 115 may extend only partially the length of the longitudinal midline. Moreover, the press-rib 115 is also shown as having a teardrop or semi-circular shape, but the press-rib 115 may be formed into other shapes as well, such as a triangle or any other convenient or desirable shape. In embodiments wherein the penis may be erect or becoming erect, as shown in FIG. 2, the press-rib 115 may be forced downward against the deep dorsal vein of the penis, thereby preventing reverse blood flow and further, maintaining the erection.

Referring back to FIG. 1, the elongated cylindrical sleeve 110 may be configured to comfortably encircle a shaft of a penis. In some embodiments, including in those wherein the penis may be flaccid, the elongated cylindrical sleeve 110 may wholly encircle the penile shaft. In other embodiments, including in those wherein the penis may be erect, the elongated cylindrical sleeve 110 may only partially encircle the penile shaft. As such, the sleeve 110 may be formed as either a full cylinder shape or a partially opened cylinder shape.

According to certain embodiments, the elongated cylindrical sleeve 110 may comprise medical-grade silicone and medical-grade polyester. In other embodiments, the sleeve 110 may be formed out of a combination of medical-grade silicone and medical-grade polyester. In still other embodiments, as understood by a person of ordinary skill, the sleeve 110 may be formed out of other medical-grade materials. In further embodiments, the elongated cylindrical sleeve 110 may comprise a gel-like material, saline, marbles, or other materials. In some embodiments, the elongated cylindrical sleeve 110 may comprise a stretchable material. For example, the stretchable material may comprise an accordion stretch material or a spring-like material. In certain further embodiments, the sleeve 110 may be smooth. In alternate embodiments, the sleeve 110 may be textured or may comprise one or more grooves, patterns, lines, shapes, symbols, or textures.

Moreover, in certain embodiments (not shown), the sleeve 110 may comprise one or more internal layers. The one or more internal layers may be formed of mesh or other materials. In such embodiments, the one or more layers may provide desirable rigidity. In alternate embodiments, the one or more layers may be formed of a stretchable material. As before, the stretchable material may comprise any stretchable material, such as the accordion stretch material or spring-like material. In other embodiments, the one or more internal layers may be formed of one or more air pockets.

Further, in some embodiments, the elongated cylindrical sleeve 110 may be formed of one or more of an antimicrobial material, an antibacterial material, or an antibiotic material. Moreover, in other embodiments, the elongated cylindrical sleeve 110 may comprise one or more antimicrobial layers or one or more antibiotic layers. In some of these embodiments, the one or more antimicrobial or antibiotic layers may be embedded or impregnated within the sleeve 110. In other embodiments, the one or more antimicrobial or antibiotic layers may be secured to one or more edges, surfaces, or layers of the sleeve 110 while the sleeve 110 is implanted within the patient's penis.

Further, while the elongated cylindrical sleeve 110 has been described as being formed as a cylinder, the sleeve 110 may vary in size, such as length and width, and shape. For instance, in certain embodiments, the size of the sleeve 110 may vary depending on the natural size and shape of the patient's penis. Alternatively, the size of the sleeve 110 may vary depending on the recommendation of the physician or the desires of the patient. Moreover, in some embodiments (not shown), the elongated cylindrical sleeve 110 may comprise a plurality of segments along its length, that is, from the proximal edge 112 to the distal edge 114. In this manner, the plurality of segments may provide further customization of the shape of the device 100 so as to suit the individual needs of the patient.

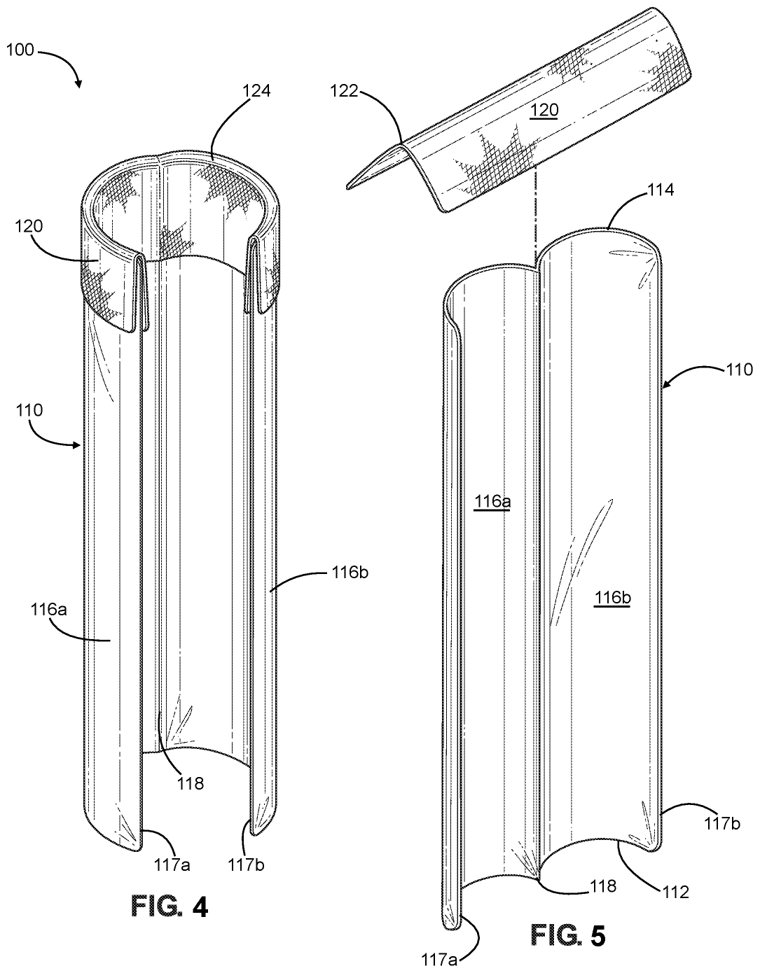

With reference now to FIGS. 4-5, the external layer 120 may be configured to cover the sleeve 110. Thus, in some embodiments, the external layer 120 and the sleeve 110 may be formed as separate pieces. In this way, the external layer 120 may be formed as a sheet or any other convenient shape. Moreover, as shown in the FIGS., the external layer 120 may be formed out of a soft mesh material so as to allow the layer 120 to easily cover the sleeve 110. For example, in some embodiments, the external layer 120 may be formed out of polypropylene, silicone, bovine tissue, marbles, or strings. One of ordinary skill in the art will appreciate that the external layer 120 may be formed out of other medically-safe, biocompatible materials as well. In other embodiments, the external layer 120 may be formed out of non-biocompatible materials and may be formed into various shapes as well.

Further, similar to the elongated cylindrical sleeve 110, the external layer 120 may be formed of one or more of an antimicrobial material, an antibacterial material, or an antibiotic material. In alternate embodiments, the external layer 120 may comprise one or more antimicrobial layers and/or one or more antibiotic layers. In certain embodiments, the one or more antimicrobial layers and/or the one or more antibiotic layers may be secured to the external layer 120 prior to or concurrent with the external layer 120 being secured to the elongated cylindrical sleeve 110. In other embodiments, the one or more antimicrobial layers and/or the one or more antibiotic layers may be secured to the external layer 120 after the external layer has been secured to the elongated cylindrical sleeve 110.

More particularly, according to some embodiments, the external layer 120 may be defined by a midline 122. As shown in FIG. 5, the midline 122 may join to and cover the distal edge 114 of the sleeve 110 so as to form a covered distal edge 124. Once the external layer 120 has covered the distal edge 114 of the sleeve 110, the covered distal edge 124 may align with a coronal sulcus of the penis. In further embodiments, the covered distal edge 124 may secure to the coronal sulcus, thereby securing the device 100 to the penis. For instance, the covered distal edge 124 may be secured to the coronal sulcus using one or more nonabsorbable surgical sutures. In certain exemplary embodiments, the one or more nonabsorbable surgical sutures may comprise polyester sutures or any other appropriate sutures, as known to those skilled in the art.

In alternate embodiments, as shown in FIG. 4, the external layer 120 and the elongated cylindrical sleeve 110 may form a single-bodied penile implant device 100. In such embodiments, the external layer 120 and the elongated cylindrical sleeve 110 may not be separate pieces. Instead, in these embodiments, the external layer 120 may be contiguously joined with the sleeve 110. Further, both or either of the external layer 120 or sleeve 110 may comprise one or more anchor points. The one or more anchor points may allow for suturing of the device 100 to the tunica albuginea of the penis.

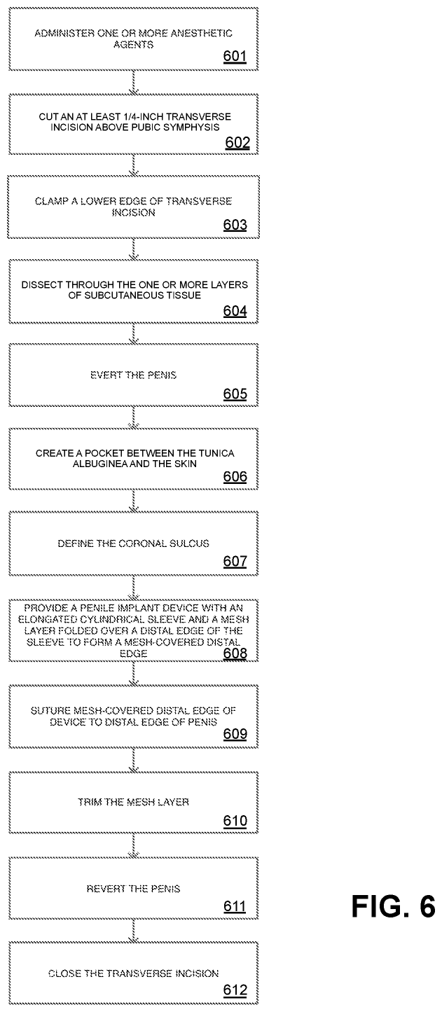

With reference to FIG. 6, one embodiment of the present invention may involve a method of implanting the aforementioned penile implant device. FIG. 6 illustrates a flowchart of one embodiment of the method of this invention. The method may improve penile appearance by cosmetically correcting conditions such as small penis perception, buried penis, micropenis, and other related diagnoses. In particular, the method may provide additional girth and length of the penis outside of the patient's body. In certain embodiments, including that demonstrated in FIG. 6, the method may comprise the steps of: administering one or more anesthetic agents to a patient having a penis (block 601); cutting an at least ¼-inch transverse incision above pubic symphysis (block 602); clamping a lower edge of the transverse incision to expose one or more layers of subcutaneous tissue (block 603); dissecting through the one or more layers of subcutaneous tissue to expose tunica albuginea (block 604); everting the penis (block 605); creating a pocket between the tunica albuginea and the skin (block 606); defining the coronal sulcus (block 607); providing a penile implant device defined by an elongated cylindrical sleeve and an external layer covering one of one or more edges, surfaces, or layers of the sleeve to form one or more covered edges, surfaces, or layers (block 608); suturing the one or more covered edges, surfaces, or layers to the penis (block 609); trimming the external layer (block 610); reverting the penis (block 611); and closing the incision (block 612).

Initially, the one or more anesthetic agents may be administered (block 601). The one or more anesthetic agents may render the patient unconscious or otherwise alter or reduce the patient's conscious state, sensation and perception of pain resulting from the procedure. In some embodiments, the one or more anesthetic agents may comprise general anesthesia or spinal anesthesia. In further embodiments, the one or more anesthetic agents may comprise general twilight anesthesia. In these embodiments, the general anesthesia may be administered in a mild dose so as to cause mild sedation and anxiolysis. In other embodiments, the one or more anesthetic agents may comprise a local anesthetic agent. For instance, the one or more anesthetic agents may be injected around the base of the penis. In such embodiments, the one or more anesthetic agents may comprise the local anesthetic agent, such as lidocaine, other long-acting local anesthetic agents, or combinations thereof.

Once the one or more anesthetic agents have been administered (block 601), the at least ¼-inch transverse incision may be cut two to three centimeters above the pubic symphysis so as to allow access to the penis (block 602). Stated differently, the at least ¼-inch transverse incision may be cut approximately one inch above the suprapubic bone. In some embodiments, the at least ¼-inch transverse incision may be 2-inches. The transverse incision may comprise an upper edge and a lower edge. Moreover, Dartos fascia may be exposed as a result of cutting the transverse incision. Indeed, in many embodiments, the transverse incision may be cut only as deep as Dartos fascia.

After the at least ¼-inch transverse incision has been cut (block 602), the lower edge of the transverse incision may be clamped to expose the one or more layers of subcutaneous tissue (block 603). One or more Allis clamps may be used to clamp the transverse incision. One or more Allis clamps may be operative to withstand and retain the weight of heavy tissue. More specifically, in some embodiments, two Allis clamps may be used to clamp the lower edge of the transverse incision. In alternate embodiments, other surgical instruments may be used to clamp, hold, or grasp the lower edge. For instance, another surgical clamp, a tenaculum, or Cushing forceps may be used to clamp the lower edge of the incision.

Next, the one or more layers of subcutaneous tissue may be dissected through to expose tunica albuginea (block 604). According to certain embodiments, scissors, an electrocautery, a combination of scissors or electrocautery may be used to dissect through the subcutaneous tissue. In other embodiments, alternate surgical dissecting instruments may be used to dissect. In further embodiments, dissecting through the one or more layers of subcutaneous tissue may further comprise releasing tissue from the suspensory ligament of the penis without removing attachment of the suspensory ligament altogether.

A pocket may be created between the tunica albuginea and the skin in order to provide space for the penile implant device (block 606). In certain embodiments, the pocket may comprise the entire length of the penile shaft from the proximal edge at the base to the distal edge at the glans penis. In others, the pocket may only comprise a partial length of the penile shaft. In some embodiments, the pocket may comprise a ¾ circumferential dissection around the penile shaft.

According to some embodiments, creating a pocket between the tunica albuginea and the skin may comprise creating a pocket between Buck's fascia and Dartos fascia. The pocket between Buck's fascia and Dartos fascia may extend the entire length of the penile shaft. In other words, the pocket may extend from a proximal edge of the penile shaft to the glans penis.

Following creation of the pocket, in some embodiments wherein the device may attach to a distal edge of the penis, the coronal sulcus may be defined (block 607). Defining the coronal sulcus of the penis may further comprise sharply dissecting the tissue around the coronal sulcus. In some embodiments, the coronal sulcus may comprise areolar tissue attached thereto. In such embodiments, defining the coronal sulcus may further comprise releasing the areolar tissue. Defining the coronal sulcus of the penis may provide access to the glans penis for subsequent suturing of the penile implant device.

In some embodiments, the elongated cylindrical sleeve and the external layer may be contiguously joined in that the sleeve and the external layer may form a single-bodied device. Alternatively, the elongated cylindrical sleeve and the external layer may be separate and may be configured to be joinable along the one or more edges, surfaces, or layers of the sleeve. For example, in certain embodiments, the external layer may be joinable along the distal edge of the sleeve. In such embodiments, providing the penile implant device (block 608) may further comprising providing the sleeve and the external layer and subsequently covering the external layer over the distal edge of the sleeve to form the covered distal edge. In still other embodiments, providing the penile implant device (block 608) may comprise providing the sleeve and the external layer, covering the external layer over the distal edge of the sleeve, and securing, such as by suturing, the external layer to the distal edge of the sleeve.

The one or more covered edges, surfaces, or layers may then be sutured to the penis (block 609). In certain exemplary embodiments, wherein the one or more covered edges, surfaces, or layers is a covered distal edge, the covered distal edge may be sutured to the coronal sulcus of the penis. Suturing the covered edges, surfaces, or layers to the edge of the penis may further comprise positioning a longitudinal midline of the penile implant device along a dorsal longitudinal midline of the penis and suturing to attach the device to the tunica albuginea. According to some embodiments, nonabsorbable surgical sutures may be used for suturing the covered edges, surfaces, or layers to the penis. In particular, the nonabsorbable surgical sutures may be secured to the tunica albuginea. In alternate embodiments, the surgical sutures may comprise polyester, silk, nylon, stainless-steel, or a combination of the foregoing. In still other embodiments, the one or more covered edges, surfaces, or layers may be sutured to the penis using absorbable surgical sutures. In certain embodiments, two or more surgical sutures may be used to secure the penile implant device to the tunica albuginea of the penis.

Finally, the external layer maybe trimmed (block 610), the penis may be reverted (block 611), and the transverse incision may be closed (block 612). Scissors or other surgical cutting instruments may be used to trim the external layer (block 610). In alternate embodiments, other instruments may be used as well. In certain exemplary embodiments, the external layer may be trimmed so as to leave a one-centimeter edge around each of the sutures. Closing the transverse incision (block 612) may comprise suturing the lower edge of the transverse incision to the upper edge of the transverse incision. In other embodiments, the lower edge of the transverse incision may be glued or otherwise secured to the upper edge of the transverse incision.

In certain further embodiments, the method may comprise one or more additional steps. In some embodiments, the method may further comprise preparing a surgical site. Preparing the surgical site may take place prior to or immediately following administering the one or more anesthetic agents. The surgical site may comprise the penis and the adjacent bodily area. Preparing the surgical site may comprise draping the surgical site with an antimicrobial drape. The antimicrobial drape may inhibit bacterial growth at the surgical site by immobilizing bacteria and providing antimicrobial activity. Preparing the surgical site may also comprise measuring preoperative penile circumference and length.

Throughout the method, in further embodiments, one or more antibiotics may be applied. Applying the one or more antibiotics may further comprising irrigating or lavaging the surgical site with the one or more antibiotics. The one or more antibiotics may comprise a triple antibiotic solution mixed with rifampicin. In other embodiments, the one or more antibiotics may comprise a triple antibiotic solution mixed with minocycline. A person of ordinary skill in the art will understand that the one or more antibiotics may comprise other known antibiotic compositions as well.

Following the method, a surgical drain may be installed. According to certain embodiments, a Jackson-Pratt drain may be installed. In other embodiments, the surgical drain may comprise a Penrose drain, a Redivac drain, negative pressure wound therapy, a Davol, a pigtail drain, or any other surgical instrument operative to remove collected fluid from the surgical site. The surgical drain may render less likely the possibility of infection and other complications resulting from commonly-occurring fluid build-up at the surgical site by removing the fluid.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### CONCLUSIONS, RAMIFICATIONS, AND SCOPE

While certain embodiments of the invention have been illustrated and described, various modifications are contemplated and can be made without departing from the spirit and scope of the invention. For example, the elongated cylindrical sleeve and the external layer may be provided separately or as a single-bodied device. As another example, the external layer may cover the one or more edges, surfaces, or layers of the elongated cylindrical sleeve. Accordingly, it is intended that the invention not be limited, except as by the appended claims.

The teachings disclosed herein may be applied to other systems and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the penile implant device and method with which that terminology is associated. In general, the terms used in the following claims should not be constructed to limit the penile implant device and method to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the penile implant device and method is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the penile implant device and method.

What is claimed is:

1. A penile implant device, comprising:
an elongated cylindrical sleeve that is configured to encircle a shaft of a penis, the elongated cylindrical sleeve defined by one or more edges, surfaces, or layers; and
an external layer configured to cover the one or more edges, surfaces, or layers of the elongated cylindrical sleeve and align with the penis.

2. The penile implant device of claim 1, wherein the elongated cylindrical sleeve is further defined by a plurality of hingedly joined sides abutting along one or more hinges.

3. The penile implant device of claim 2, wherein the plurality of sides comprise a first side and a second side abutting along a hinge.

4. The penile implant device of claim 3, wherein the one or more edges, surfaces, or layers comprise a distal edge configured to align with a coronal sulcus of the penis, a proximal edge configured to align with a base of the penis, a first lateral edge disposed along the first side opposite the hinge, a second lateral edge disposed along the second side opposite the hinge, a top surface, and a bottom surface.

5. The penile implant device of claim 4, wherein the external layer covers the distal edge of the elongated cylindrical sleeve to form a covered distal edge configured to align with the coronal sulcus of the penis.

6. The penile implant device of claim 3, wherein the elongated cylindrical sleeve further comprises a press-rib, and wherein the first side and the second side rotate around the hinge outwardly and the press-rib presses a deep dorsal vein of the penis during penile erection.

7. The penile implant device of claim 3, wherein the first side and the second side rotate around the hinge inwardly during penile flaccidity.

8. The penile implant device of claim 1, wherein the elongated cylindrical sleeve is formed of a gel-like material, saline, marbles, or one or more internal layers formed of one or more of medical-grade silicone, polyester, and one or more air pockets.

9. The penile implant device of claim 1, wherein the elongated cylindrical sleeve and the external layer are contiguously joined.

10. The penile implant device of claim 1, wherein the elongated cylindrical sleeve and the external layer are separate and are configured to be joinable along the one or more edges, surfaces, or layers of the sleeve.

11. The penile implant device of claim 1, wherein the external layer is formed of polypropylene, silicone, bovine tissue, or other biocompatible material.

12. A penile implant method, comprising the steps of:
administering one or more anesthetic agents to a patient having a penis;
cutting an at least ¼-inch transverse incision above pubic symphysis;
clamping a lower edge of the transverse incision to expose one or more layers of subcutaneous tissue;
dissecting through the one or more layers of subcutaneous tissue to expose tunica albuginea;
everting the penis;
creating a pocket between the tunica albuginea and the skin;
providing a penile implant device defined by an elongated cylindrical sleeve and an external layer covering one or more edges, surfaces, or layers of the sleeve to form one or more covered edges, surfaces, or layers;
suturing the one or more covered edges, surfaces, or layers to the penis;
trimming the external layer;
reverting the penis; and
closing the incision.

13. The penile implant method of claim 12, wherein the anesthetic agent comprises at least one of a general anesthetic agent, a general twilight anesthetic agent, a spinal anesthetic agent, and a local anesthetic agent.

14. The penile implant method of claim 12, wherein dissecting through the one or more layers of subcutaneous tissue to expose tunica albuginea further comprises
electrocauterizing and cutting the one or more layers of subcutaneous tissue; and
releasing subcutaneous tissue from the suspensory ligament.

15. The penile implant method of claim 12, wherein the elongated cylindrical sleeve and the external layer are contiguously joined.

16. The penile implant method of claim 12, wherein the elongated cylindrical sleeve and the external layer are separate and are configured to be joinable along one or more edges, surfaces, or layers of the sleeve.

17. The penile implant method of claim 12, wherein the external layer covers a distal edge of the elongated cylindrical sleeve and the one or more covered edges, surfaces, or layers comprise a covered distal edge.

18. The penile implant method of claim 12, wherein suturing the one or more covered edges, surfaces, or layers to the penis further comprises
positioning a longitudinal midline of the penile implant device along a dorsal longitudinal midline of the penis; and
suturing two or more surgical sutures to attach the device to the tunica albuginea.

19. The penile implant method of claim 18, wherein the two or more surgical sutures are polyester sutures.

20. A penile implant device, comprising:
an elongated cylindrical sleeve that is configured to encircle a shaft of a penis, the elongated cylindrical sleeve defined by a proximal edge, a distal edge, a first side, a second side, and a hinge, wherein the first side and the second side abut along the hinge; and
an external layer covering the distal edge of the elongated cylindrical sleeve to form a covered distal edge, the covered distal edge configured to align with a coronal sulcus of the penis.
